SEQUENCE LISTING (20 sequences)

SEQ ID NO: 1 - DNA, 197 bp, Homo sapiens
atgggccgcgcgggcgcaggctcccccgggcgccgcaggcagcggtgccagagccggggcaggcggcggccgcgagcccctcggcggcggaaggccccagcgtgcaggcgcaggagggcgcggcgccggcggaagaagccctgtccccgcagcttgcgaccggagatccacgaatgtcccaagtcccaggacccggt

SEQ ID NO: 2 - DNA, 251 bp, Mus ssp.
atgggccgcgcggccgcccgcaagcggggacgctgcagacagcgcggcggatccccgagaggccggcgacgccgtggacctggacgccaaagtcctaggaaacgcccgggccctcggcgaaggaaagcgcgcgcgcgccgccgcaggagggcgcgccctcgccggatggagcccattcctgaacctttcaacccggggcctctgctgcaagagcctccccagtactgcaacagctcgagttcctgggcttg

SEQ ID NO: 3 - DNA, 274 bp, Rattus ssp.
atgggccgcgcggccgcccggaagccgggccgccagtgtgctggaattcgcccttatgggccgcgcggccggtccccgggaggccggcgacggcgtgaacctggacgccaaagtcctaggaagcgcccaggccctcggaggaggagagctcgcgcgcgccgccgcaggagggcgcgccctcgccggatggagcccattcccgagcctttcaacccgcggcctctgctgcaggaccttccccagaccagcaacagccacgagttcctgggcttgt

SEQ ID NO: 4 - DNA, 231 bp, Sus scrofa
atgtccgaagacgggtgggcatgggccgcaccagccgccggttccccgggtcgccgtggccagcggcgccggttgccggccgggcggcgacgcagggcccctcggaggcggagggctcgtttgtgccgcagaagggcgcgcccccggagaaggcagccggcttccgagagcctgggcagggggggcccgcggccggggagagcggctgcagcgccgagtcccaggacccgg

SEQ ID NO: 5 - DNA, 27 bp, Homo sapiens
gtcacagtcgcgcagtcctgactacgg

SEQ ID NO: 6 - DNA, 27 bp, Homo sapiens
cctgctgaccgcgacacgcgcgaggta

SEQ ID NO: 7 - protein, 65 aa, Homo sapiens
MGRAGAGSPGRRRQRCQSRGRRRPRAPRRRKAPACRRRRARRRRKKPCPRSLRPEIHECPKSQDP

SEQ ID NO: 8 - protein, 83 aa, Mus ssp.
MGRAAARKRGRCRQRGGSPRGRRRRGPGRQSPRKRPGPRRRKARARRRRRARPRRMEPIPEPFNPGPLLQEPPQYCNSSSSWA

SEQ ID NO: 9 - protein, 91 aa, Rattus ssp.
MGRAAARKRGRQCAGIRPYGPRGRSPGGRRRREPGRQSPRKRPGPRRRRARARRRRRARPRRMEPIPEPFNPRPLLQDLPQTSNSHEFLGL

SEQ ID NO: 10 - protein, 77 aa, Sus scrofa
MSEDGWAWAAPAAGSPGRRGQRRRLPAGRRRRAPRRRRARLCRRRARPRRRQPASESLGRGGPRPGRAAAAPSPRTR

SEQ ID NO: 11 - DNA, 28 bp, Artificial sequence
  Description of artificial sequence oligonucleotide primer PHGPx Ea-fl
gggacgctgcagacagcgcggcggatcc

SEQ ID NO: 12 - DNA, 16 bp, Artificial sequence
  Description of artificial sequence oligonucleotide primer 1 fur Ratten snGPx
atgggccgcgcggccg

SEQ ID NO: 13 - DNA, 24 bp, Artificial sequence
  Description of artificial sequence oligonucleotide primer 2 fur Ratten snGPx
cggcaggtccttctctatcacctg

SEQ ID NO: 14 - DNA, 25 bp, Artificial sequence
  Description of artificial sequence oligonucleotide primer 1 fur humanes snGPx
atgggccgcgcgggcgcaggctccc

SEQ ID NO: 15 - DNA, 28 bp, Artificial sequence
  Description of artificial sequence oligonucleotide primer 2 fur humanes snGPx
cttgcgaccggagatccacgaatgtccc

SEQ ID NO: 16 - DNA, 31 bp, Artificial sequence
  Description of artificial sequence oligonucleotide primer GFP-Ea-f1
gatctctagaccggcgggcatgggccgcgcg

SEQ ID NO: 17 - DNA, 39 bp, Artificial sequence
  Description of artificial sequence oligonucleotide primer GFP-Ea-r2
gctcctcgcccttgctcaccaagcccaggaactcggagc

SEQ ID NO: 18 - DNA, 31 bp, Artificial sequence
  Description of artificial sequence oligonucleotide primer GFP-Ea-f2
gatctcagactcgccggatggagcccattcc

SEQ ID NO: 19 - DNA, 39 bp, Artificial sequence
  Description of artificial sequence oligonucleotide primer GFP-F3
gctccgagttcctgggcttggtgagcaagggcgaggagc

SEQ ID NO: 20 - DNA, 20 bp, Artificial sequence
  Description of artificial sequence oligonucleotide primer GFP-r1
cctctacaaatgtggtatgg

(19) United States
(12) Patent Application Publication
(10) Pub. No.: US 2004/0219562 A1
(43) Pub. Date: Nov. 4, 2004

(54) Novel form of the PHGPx protein as a diagnostic marker for male infertility

(76) Inventors: Dietrich Behne, Berlin (DE); Georg Bornkamm, Munchen (DE); Markus Brielmeier, Garching (DE); Henning Pfeifer, Berlin (DE); Antonios Kyriakopoulos, Berlin (DE); Marcus Conrad, Munchen (DE); Jorg Schmidt, Munchen (DE)

Correspondence Address: JENKINS & WILSON, PA, 3100 TOWER BLVD, SUITE 1400, DURHAM, NC, 27707, US

(21) Appl. No.: 10/636,796

(22) Filed: Aug. 7, 2003

Related U.S. Application Data
(63) Continuation of application No. PCT/EP02/01648, filed on Feb. 15, 2002.

(30) Foreign Application Priority Data
Feb. 15, 2001 (DE) 10107186.8

(51) Int. Cl. C12Q 1/68; C07H 21/04; C12N 9/08

(52) U.S. Cl. 435/6; 435/69.1; 435/192; 435/320.1; 435/325; 536/23.2

(57) ABSTRACT

This invention relates to a novel form of the PHGPx protein, the sperm nuclei glutathione peroxidase (snGPx) as well as portions thereof playing a role in mammalian spermatogenesis, and to the nucleic acids encoding the same. The invention further relates to vectors containing said nucleic acid and to host cells transformed by these vectors. Furthermore the invention comprises antibodies specific for the above proteins/peptides as well as the use of the proteins/peptides in the diagnosis or therapy of male infertility.

### RELATED APPLICATIONS

This application is a continuation of PCT patent application No. PCT/EP02/01648, filed Feb. 15, 2002, which claims priority to German patent application No. 10107186.8, filed Feb. 15, 2001, the disclosures of each of which are incorporated herein by reference in their entirety.

### TECHNICAL FIELD

This invention relates to a novel form of the PHGPx protein, the sperm nuclei glutathione peroxidase (snGPx) as well as portions thereof playing a role in mammalian spermatogenesis as well as to the nucleic acids encoding the same. Furthermore, the invention is directed to vectors containing these nucleic acids and host cells transformed by these vectors. Moreover, the invention comprises antibodies specific for the above-mentioned proteins/peptides and also the use of the proteins/peptides in the diagnosis or therapy of male infertility.

### BACKGROUND ART

The phospholipid hydroperoxide glutathione peroxidase (PHGPx, also called GPx-4) is a selenoenzyme. It carries selenocysteine as the 21^{st} amino acid in the active site and is an oxido-reductase which is capable of detoxifying H₂O₂. In the cell, it is present in the cytosol and in the mitochondria, is expressed ubiquitously and has the unique feature that in contrast to other glutathione peroxidases it can also reduce lipid peroxides, oxidized LDL, and oxidized cholesterol. To complete the reaction, glutathione is oxidized. Another unique feature of PHGPx is that during a glutathione deficiency when no glutathione thiol groups are available as the electron source also protein thiols can be used as substrates and thus disulfide bridges can be introduced into proteins.

Several studies show that selenium plays an important role in the sperm development of mammals. For rats the selenium concentration has been found to markedly increase within the testes following the onset of adolescence and it has been found that sperms are the main target cells for the element and by far show the highest selenium levels of all compartments in the rat (1). Selenium-depleted rats produced sperms with inferior motility and abnormalities in the central portion (2). Similarly, in selenium-deficient mice abnormalities were observed which most frequently consisted of deformities of the sperm head (3). A severe depletion of selenium in rats caused testicular atrophy and a complete interruption of spermatogenesis (4).

An in vivo labeling of rats with ⁷⁵Se and separation of the tissue homogenates by gelelectrophoretic methods showed that within the organism the element is present in a major amount of selenium-containing proteins (5, 6). Among these, a protein having an apparent molecular weight of about 34 kD is found exclusively in testes and sperms (5). It is located in the nuclei of the sperm cells an is expressed to a high level in the late spermatids (7). During these stages the nuclei undergo significant modifications characterized by a replacement of the histones by the protamines and the reorganization and condensation of DNA governed by crosslinking of the protamine thiols and leading to a structure which is highly resistant to chemical and mechanical stress (8).

Previously, the PHGPx enzyme has been attributed only a function as a protective enzyme against oxidative damage. However, one and a half years ago, Flohe and Ursini (29) have demonstrated that the same enzyme in an oxidized form (i.e. with inter- and intramolecularly crosslinked disulfide bridges) is involved in forming the mitochondrial capsule of sperms. Large amounts of the enzyme are required in sperms for this purpose.

Besides PHGPx, however, there exist a plurality of selenium-containing proteins as described above the structure and function of which in male fertility are still unknown.

### SUMMARY OF THE INVENTION

Therefore, the object underlying the present invention is to provide a novel selenoprotein representing an important marker of male fertility.

A further object underlying the present invention is to provide novel methods for in vitro diagnosis and therapy of male infertility.

The determination of the structure of the selenoprotein according to the invention and the nucleic acids encoding said selenoprotein, respectively, is an important contribution to the in vitro diagnosis of male infertility as well as to therapies of male infertility.

Presently disclosed is a novel selenoprotein and the nucleic acid sequence on which this selenoprotein is based. After purification and sequencing, the inventors have isolated a selenoprotein which is a novel form of PHGPx generated by alternative splicing of the PHGPx RNA. This novel and alternative form of PHGPX (snGPx) is expressed only in testis where it is very strongly expressed. The alternative exon expressed in the novel splicing product is strongly basic and mediates the nuclear localization of the protein. Due to its biochemical properties it thus seems very likely that this enzyme is responsible for nuclear condensation which itself is caused by inter- and intramolecular crosslinking of the thiol groups in protamines. The alternative exon shows a high degree of sequence homology with respect to protamines and presumably binds directly to DNA via the strongly basic amino acids. It has been found that nuclear condensation can be reversed by high concentrations of dithiothreitol (DTT). If rats are fed with a selenium-depleted diet they form much lower amounts of the novel selenoprotein and exhibit a defect in nuclear condensation of their sperms. This also is a reason for male infertility in rats under selenium depletion. The causal connection between the novel form of PHGPx and nuclear condensation thus is compelling with respect to function.

Principally the novel selenoprotein is encoded by a nucleic acid sequence containing exons 2-7 of the PHGPx gene and an alternative exon within the first intron of the PHGPx gene. In addition the nucleic acid may optionally contain exon 1 of the PHGPx gene. The genomic sequence as well as the coding regions of the human PHGPx gene are already known from (41). The human selenoprotein PHGPx (also called GPx-4) is encoded by 7 exons comprising bases 2663-2746 (exon 1), 3806-3900 (exon 2), 3986-4130 (exon 3), 4278-4429 (exon 4), 4863-4887 (exon 5), 5021-5080 (exon 6) and 5161-5193 (exon 7) of the genomic sequence.

The exons 1-7 of the PHGPx gene are highly conserved sequences which have been already described for several mammalian species (mouse: (39), pig: (40), man: (41, supra)).

However, it has been surprisingly found that the N-terminal amino acid sequences of the novel selenoprotein are encoded by a so far unknown alternative exon which is only weakly conserved between different mammalian species. As detailed above, this alternative exon and the protein encoded thereby has a surprising function in mediating the nuclear localization of the selenoprotein. Thereby, it exerts an important function in the allover function and in male infertility as a whole. In the human genomic sequence the alternative exon comprises bases 3355-3551, and consequently is localized between the first and the second exons of the PHGPx selenoprotein known up to now (or, in other words, in the first intron of the PHGPx gene).

According to one embodiment of the invention the nucleic acid encodes a human selenoprotein wherein the alternative exon comprises the sequence represented in SEQ ID NO: 1 or a portion thereof which codes for a biologically active peptide.

Other alternative exons for mouse, rat, and pig are defined in SEQ ID NOs: 2-4.

The term alternative exon as used herein means an exon which is for example formed by differential splicing of a single RNA primary transcript. A “portion” of the nucleic acids according to the invention is intended to mean particularly derivatives and fragments encoding a biologically active peptide. Biologically active in this respect means that the variations code for a selenoprotein wherein the natural function is preserved, i.e. which is capable of maintaining male fertility of mammals.

Derivatives of the above nucleic acids are for example such nucleic acids having one or more substitutions, insertions and/or deletions as compared to the respective sequence of SEQ ID NOs: 1-4 wherein the derivative binds to the respective nucleic acid according to SEQ ID NOs: 1-4 under moderately stringent or stringent conditions. Derivative according to the invention particularly means those nucleic acids wherein at least 1, but also 2, 3, 4 or more nucleotides have been deleted or replaced by other nucleotides at one or both ends of the nucleic acids or also in the central portions of the nucleic acids.

Thus, the nucleic acids of the present invention also comprise nucleic acids having sequences which are essentially equivalent to the nucleic acids according to SEQ ID NOs: 1-4. Nucleic acids according to the invention may have e.g. at least about 80%, generally at least about 90% or 95% sequence identity to the nucleic acids according to SEQ ID NOs: 1-4. A provision for this is, however, in each case that the variations comply with the above defined biological function of the selenoprotein. Furthermore, the invention provides complementary sequences to the above mentioned nucleic acids.

The term “nucleic acid sequence” refers to a heteropolymer of nucleotides or the sequence of these nucleotides.

The term “nucleic acid”, as used herein, comprises RNA, DNA including cDNA, genomic DNA and synthetic (for example chemically synthesized) as well as bases bound to other polymers, such as PNA.

The invention comprises—as mentioned above—also such derivatives hybridizing to the nucleic acids according to the invention under moderately stringent or under stringent conditions.

Stringent hybridization and washing conditions generally means the reaction conditions under which only duplex molecules between oligonucleotides and the desired target molecules (perfect hybrids) are formed and only the desired target organism is detected, respectively. Stringent hybridization conditions particularly are 0.2 ×SSC (0.03 M NaCl, 0.003 M sodium citrate, pH 7) at 65° C. In the case of shorter fragments, for example oligonucleotides consisting of up to 20 nucleotides, the hybridization temperature is lower than 65° C., for example more than 55° C., preferably more than 60° C. but in each case lower than 65° C. Stringent hybridization conditions depend on the size or length of the nucleic acid and its nucleotide compositions and may be determined by those skilled in the art by manual experimentation. Moderately stringent conditions are for example achieved at 42° C. and washing in 0.2 ×SSC/0.1% SDS at 42° C.

The respective temperature conditions may be different depending on the experimental conditions selected and depending on the nucleic acid sample to be examined, and in this case have to be adjusted accordingly. The detection of the hybridization product may be for example performed using autoradiography for radiolabeled moleculers or by fluorimetry if fluorescence-labeled molecules are used.

Those skilled in the art in a manner known per se are able to adapt the conditions to the method of examination selected to actually achieve stringent conditions and to enable a specific detection process. Suitable stringent conditions may be determined for example using reference hybridizations. An appropriate concentration of nucleic acid or oligonucleotide, respectively, must be employed. The hybridization has to be carried out at the appropriate temperature.

According to further embodiments, the present invention comprises a mammalian selenoprotein encoded by the nucleic acid according to claim 1. This may preferably be an N-terminal sequence as defined in SEQ ID NOs: 7-10 or homologs or fragments thereof retaining a biological activity.

Furthermore, the invention comprises peptides according to SEQ ID NOs: 7-10 or homologs or fragments thereof retaining a biological activity.

It is obvious for those skilled in the art of the field to which the invention belongs that the practice of the present invention is not limited to the use of the particular sequences defined in SEQ ID NOs: 1-10.

Modifications of the sequences such as for example deletions, insertions or substitutions within the sequence which generate so-called “silent” changes in the protein molecule obtained are also considered as falling within the scope of the present invention.

As an example, changes in the nucleic acid sequence are considered which result in the generation of an equivalent amino acid at the given site.

Preferably, such amino acid substitutions are the result of a substitution of one amino acid by another amino acid having similar structural and/or chemical properties, i.e. conserved amino acid substitutions. Amino acid substitutions may be performed due to the similarity in polarity, charge, solubility, hydrophobicity, hydrophilicity and/or the amphipathic (amphiphilic) nature of the residues involved. Examples of apolar (hydrophobic) amino acids are alanine, leucine, isoleucine, valine, proline, phenylalanine, tryptophane, and methionine. Polar neutral amino acids include glycine, serine, threonine, cysteine, tyrosine, asparagines, and glutamine. Positively charged (basic) amino acid include arginine, lysine, and histidine. And negatively charged (acidic) amino acids include aspartic acid and glutamic acid.

“Insertions” or “deletions” typically cover a range of one to five amino acids. The permissible degree of variation may be determined experimentally by systematically performed insertions, deletions or substitutions of amino acids within a peptide molecule using DNA recombination techniques and by examining the resulting recombinant variations with respect to their biological activity.

Nucleotide changes resulting in an alteration of the N-terminal and C terminal portions of the protein molecule often do not alter the protein activity because these portions usually are not involved in biological activity. It can be also desired to eliminate one or more cysteines present in the sequence because the presence of cysteines may result in an undesired formation of multimers if proteins are produced in a recombinant manner thus complicating the purification an crystallization procedures. Each of the modifications suggested is within the range of the technical and natural scientific basic knowledge as is the determination of the activity retained by the encoded proteins.

Accordingly, where the term “DNA sequence” is used either in the description or in the claims, it is intended to comprise all such modifications and variations resulting from the preparation of a biologically equivalent peptide/protein.

Furthermore, the present invention relates to an expression vector containing one of the nucleic acid sequences referred to above.

A plurality of vectors suitable for use in transforming bacterial cells are known, for example plasmids and bacteriophages such as phage λ may be used being the most commonly used vectors for bacterial hosts. Both in mammalian and in insect cells viral vectors may be used for example for the expression of an exogenous DNA fragment. Exemplary vectors are the SV40 and the polyoma virus.

The transformation of the host cell may be alternatively performed by “naked DNA” without using a vector.

According to a preferred embodiment of the invention the nucleic acids according to SEQ ID NOs: 1-4 and fragments, transcripts or derivatives thereof, respectively, are hybridized as probes with the DNA or RNA sample to be studied using stringent or moderately stringent hybridization conditions. The conditions of stringency must be determined empirically for each application. The conditions described above may be used as guidelines.

The term “probe” as defined herein means a nucleic acid capable of binding to a target nucleic acid of a complementary sequence by means of one or more types of chemical bonds, typically by pairing of complementary bases which generally takes place via hydrogen bridges.

The generation of a fusion protein according to the invention may be performed either in eukaryotic cells or in prokaryotic cells. Examples of suitable eukaryotic cells include mammalian cells, plant cells, yeast cells, and insect cells. Suitable prokaryotic hosts includes E. coli and Bacillus subtilis.

According to one embodiment of the invention a method is provided for the generation of an essentially pure selenoprotein/peptide comprising transforming of a host cell by a vector according to claim 22, culturing the host cell under conditions enabling an expression of the sequence by the host cell, and isolating the selenoprotein/peptide from the host cell.

The proteins/peptides according to the invention are also serologically active, immunogenic and/or antigenic. Therefore, they may be further used as immunogens for the preparation of both polyclonal and monoclonal specific antibodies.

These specific antibodies may be used in diagnosis/prognosis. The selenoprotein/peptide-specific antibodies may be for example used in laboratory diagnostics using immunofluorescence microscopy or in immunohisto-chemical staining or as a component in immunoassays for the detection and/or quantification of the selenoprotein/peptide in clinical samples. Such specific antibodies may be used as components of diagnostic/prognostic kits. Moreover, such antibodies may be used in the affinity purification of the selenoproteins/peptides of the invention.

Preferably, an antibody directed against the amino acid sequence defined by the alternative exon will be produced which enables a specific immunohistochemical measurement of snGPx besides PHGPx.

Furthermore, the invention relates to a composition containing a hybridoma having a monoclonal antibody with a binding specificity against one of the proteins/peptides disclosed.

A monoclonal antibody specific for the selenoproteins/peptides according to the invention may be prepared as follows. A mouse is injected twice with the selenoprotein/peptide according to the invention. The first injection contains the selenoprotein/peptide according to the invention in complete Freund's adjuvant and is administered subcutaneously. The second injection contains the selenoprotein/peptide according to the invention in incomplete Freund's adjuvant and is administered intraperitoneally. Several injections will follow at different time points over a period of several months. Then, spleen cells are obtained from the mouse and fused to myeloma cells in polyethylene glycol. The resulting hybridoma cells are then screened to determine which produces the antibody showing the desired specificity (according to Milstein, C., Monoclonal Antibodies, Scientific American).

Furthermore, the present invention comprises a method of screening for the in vitro determination of the fertility of a mammal comprising the following steps: sperm DNA is isolated, the alternative exon of the PHGPx gene is amplified by means of PCR, the gene segments amplified are sequenced, and the matching of the gene segments amplified with the nucleic acid of an alternative exon according to the invention is monitored. A nonmatch of the sequences, for example in the form of insertions/deletions/substitutions within the sequence examined, is then an important indication that the mammal in question suffers of a fertility disorder/infertility which is caused by insufficient/lack of expression of the alternative exon. Based on this result, the deficient/missing selenoprotein according to the invention may be specifically administered in the context of an in vitro/in vivo therapy (see below).

In preferred embodiments, the determination for humans, mouse, rat, and pig each is performed with respect to the nucleic acid sequences given in SEQ ID NOs: 1-4. However, it should be understood that the invention is not limited to these animal species. In contrast, it may be used advantageously in the determination of fertility (and, in the case of a negative sequence match also in the determination of infertility) of all species of agricultural and domestic animals. Such agricultural and domestic animals are for example cattle, horses, sheep, goats, cats, and dogs.

According to a preferred embodiment, first a determination of nuclear condensation is performed by staining with acridine orange, Feulgen's reagent or acidic aniline blue and microscopic detection. Condensed sperm nuclei are resistant to the incorporation of dyes (Feulgen's reagent, aniline blue) and resistant to a denaturation of the DNA by heat or by an acidic medium. At a specific UV excitation, acridine orange stains double stranded DNA green, while single stranded DNA and RNA is stained red. Due to a disturbed nuclear condensation the DNA can be denatured by acid (fixation in acidic alcohol) or heat. In this case, acridine orange stains the nucleus red. The DNA of condensed nuclei cannot be denatured and shows green fluorescence. Using these methods, patient samples with disturbed condensation of the sperm head may be detected. This preselection enables a specific selection of such sperm samples which are then used in the more complex screening method.

Several reasons may underlie a lack or suboptimal amount of snGPx and the infertility caused thereby:

1. Selenium deficiency. Therefore, the selenium state of the patient should be determined.

2. Other substances affecting the activity of snGPx such as environmental chemicals and heavy metals.

3. A genetic defect causing a defective alternative splicing.

4. A mutation within the sequence in the region of the localization signal so that despite of sufficient amounts of snGPx it is not transported into the nucleus. This effect may occur already with very minor sequence changes.

5. Other defects in the sequence and thus in the function of snGPx.

Consequently, a lack of the alternative form of PHGPx results in a selective defect in sperm nucleus condensation whereas all other functions of PHGPx of protection of the DNA against oxidative stress are not affected. It became clear from animal experiments performed by the inventors that already a lack in one allele in the classical form of PHGPX results in a defective spermatogenesis and formation of the mitochondrial sperm capsule. With respect to inheritance these mutations are eliminated immediately due to the lack of progeny and thus up to now could not be recognized in genetic studies. Only a specific search for mutations in the PHGPx gene on both alleles of the genome of infertile male individuals showing defective sperm nucleus condensation can elucidate the reason for this form of infertility.

By means of the detection of the nuclear condensation in sperm it is possible to identify infertile male patients for which an IVF (in vitro fertilization) is not suitable. For these patients an ICSI (intracytoplasmic spermia injection) is therefore indicated. The additional detection in these patients of mutations in the PHGPx gene improves the diagnosis of male infertility and optionally also provides insights as to its genetic basis.

The combination of these morphological and molecular detection methods enables an identification and classification of these patients, improves the diagnosis and opens novel prognostic and therapeutic possibilities.

The invention further relates to a recombinant non-human mammal wherein the DNA sequence according to claim 1 has been inactivated. Preferably, a recombinant mouse is provided wherein the nucleic acid according to SEQ ID NO: 2 has been inactivated. Thus, using such a recombinant knock out mouse an animal model may be established.

These animal models provide novel insights into the etiology of several disorders associated with infertility of male mammals.

Eventually, the present invention comprises compositions comprising an effective amount of a protein/peptide according to any of the claims 13-21 in combination with a pharmaceutically acceptable carrier as well as the use thereof in the in vitro diagnosis of male infertility and in the in vitro/in vivo therapy of male infertility.

The protein according to the invention may be used for example in vivo in the case of disorders of male infertility if the infertility is caused e.g. by one of the following defects: (a) a genetic defect resulting in a deficiency in alternative splicing; (b) a mutation in the sequence in the region of the localization signal so that despite of sufficient amounts of snGPx the selenoprotein is not localized in the nucleus. This effect may arise already from minor sequence changes; or (c) other defects in the sequence and thus in the function of snGPx.

The administration of the protein according to the invention in vivo is preferably carried out by direct injection into the testicle. The protein may also be used therapeutically in vitro, e.g. in the treatment of sperm intended for IVF.

### BRIEF DESCRIPTION OF THE DRAWINGS

FIG. 1: selenoprotein in CTAB-treated sonification resistant nuclei of late spermatids (SRS/CTAB nuclei) and of epididymal sperms (SRSP/CTAB nuclei). Following in vivo labeling of rats with ⁷⁵Se, isolation of the nuclei by SDS PAGE, the selenoproteins transferred to a PVDF membrane were determined by means of an immunereaction with an anti-PHGPx antibody or by autoradiography of the ⁷⁵Se tracer.

FIG. 2: Differences in the isoelectric point of the selenoproteins present in the sperm nucleus partially caused by processing of snGPx. The proteins of CTAB-treated epididymal sperm nuclei were separated by two-dimensional electrophoresis and the ⁷⁵Se labeled selenoproteins determined by autoradiography.

FIG. 3: Composition of the alternative exon Ea encoding the N-terminal sequence of the sperm nucleus glutathione peroxidase (snGPx). A) Differences in the primary structure of PHGPx and snGPx: PHGPx is coded for by the seven exons E1 to E7 (28) while snGPx is encoded by exons Ea to E7 due to alternative splicing. B) Sequence of Ea and the corresponding N-terminal sequence of snGPx in mouse, rat, human and pig.

FIG. 4: Distribution of snGPx and PHGPx in the murine tissues as determined by Northern blot analysis: 20 μg of total RNA were applied per lane, blotted onto a membrane and probed with the coding region of the alternative exon (1) or with the complete PHGPx cDNA (2).

FIG. 5: Effect of selenium deficiency on the sperm nuclear concentration in rat. Sperms of rats supplied with adequate amounts of selenium (a) and of seleniumdeficient animals (b) were collected from the vas deferens and stained with acridine orange. Acridine orange stains double stranded DNA green and single stranded DNA red. Because sperm DNA is only acid resistant if the protamines are crosslinked by disulfides, the method enables the determination of chromatin condensation and of the protamine disulfide status. Almost all sperm nuclei of selenium-deficient rats show abnormal condensation.

FIGS. 6A and B: SCSA of a male volunteer aged 37, efertile, healthy.

FIGS. 7A and B: SCSA of an anonymous male volunteer, infertile.

### DETAILED DESCRIPTION OF THE INVENTION

A 34 kD selenoprotein isolated from rat testes was identified as a specific sperm nuclei glutathione peroxidase (snGPx) having properties similar to those of PHGPx. The determination of its primary structure by analysis of the first N-terminal amino acids, a data base search, polymerase chain reaction, and sequencing of the cDNA showed that its N-terminal sequence is different from that of PHGPx. This sequence encoded by an alternative exon in the first intron of the PHGPx gene shows more than 50% homology to the protamine sequences and contains a nuclear localization signal. In rats, snGPx is highly expressed in the nuclei of late spermatids where it is the only selenoprotein present. Its appearance coincides with DNA rearrangements resulting in highly condensed chromatin stabilized by crosslinked protamine thiols. In selenium-depleted rats in which the concentration of snGPx has been reduced to one third of the normal level the chromatin condensation is severely defective. We have shown that snGPx acts as a protamine thiol peroxidase responsible for disulfide crosslinking by reduction of reactive oxygen species. Its dual function in chromatin condensation and in the protection of sperm DNA from oxidation is necessary to ensure male fertility and sperm quality.

Identification of the 34 kD Selenoprotein

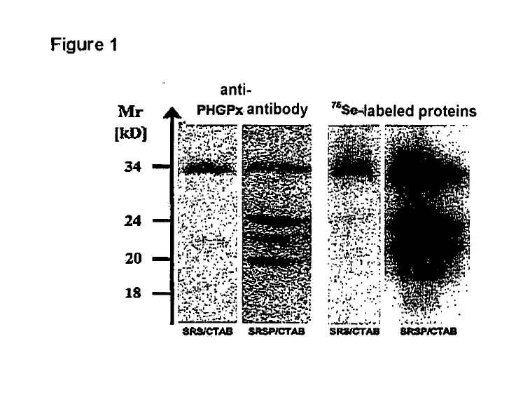

In a first step the 34 kD selenoprotein was purified from the SRS nuclei of late spermatids of rats which had been fed a selenium-adequate diet. Following treatment of the surfaces of these nuclei with the detergent CTAB it was found that the 34 kD protein was the only selenoprotein within these nuclei as can be seen from the autoradiograph in FIG. 1, lane 3. For the selenium concentration and thus for the 34 kD selenoprotein in CTAB treated SRS nuclei a relatively high value of about 60 μmol/kg of dry weight was determined which far exceeded the selenium concentration in the nuclear fractions of other rat tissues such as for example the liver (14 μmol Se/kg of dry weight) and of brain (8 μmol Se/kg of dry weight).

A protein fragment analysis by means of MALDI-MS after tryptic digestion showed a considerable similarity between the 34 kD protein and another selenoprotein, namely PHGPx, one of the four glutathione peroxidases identified up to now which catalyze the reduction of peroxide by oxidation of glutathione (12, 15-17).

The similarity became more striking in an experiment by finding out that the 34 kD protein reacts with an anti-PHGPx antibody as represented in the first lane of FIG. 1. It also inhibited the glutathione peroxidase activity and, similar to PHGPx, catalyzed the reduction of various peroxides such as for example hydrogen peroxide, t-butylhydroperoxide and phosphatidylcholine hydroperoxide (12). Its specific activity was about 3000 U/mg protein when the latter was used as the substrate. Due to its localization this was called sperm nuclei glutathione peroxidase (snGPx).

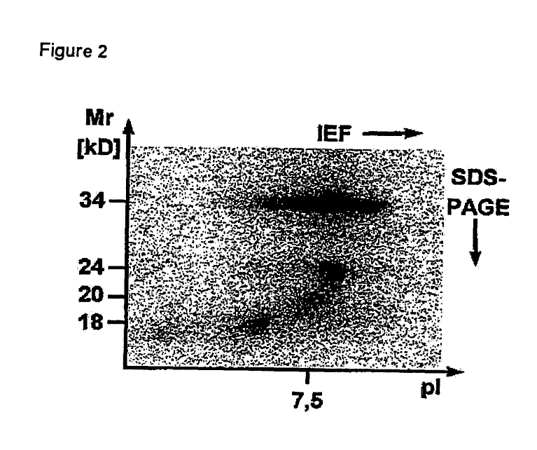

During the transfer of the sperms from the testicle to the epididymis two thirds of the 34 kD selenoprotein were converted to smaller proteins having molecular weights of 24, 22 and 20 kD, as may be seen from lanes 2 and 4 in FIG. 1. This truncation is associated with a shift in the pH value from an extremely basic value of more than 10 for the 34 kD protein to a value of about 7.5 for the 20 kD product (FIG. 2). Reduction of the mass, however, had no effect on the enzymatic properties of the processed proteins.

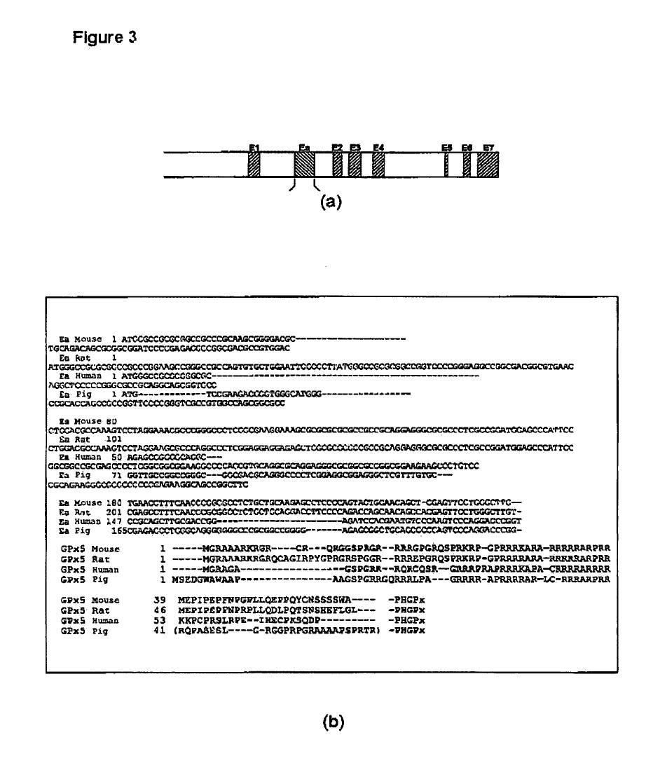

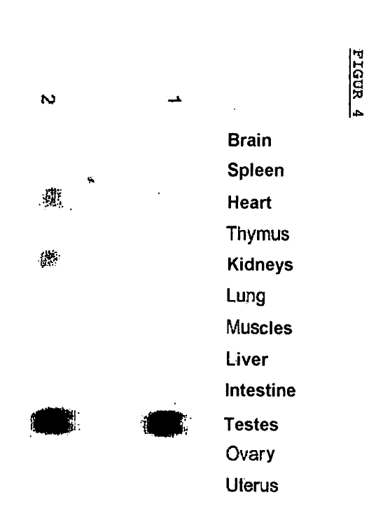

The analysis of the N-terminal sequence of the 34 kD compound showed that it started with the amino acids SRAAARGRKR. Data base searches revealed that the protein was unknown up to now. However, if the search was extended to all available genomic sequences and these were transformed into all reading frames we ended up with a similar sequence in the first intron of the murine PHGPx gene. An alternative exon responsible for the formation of the novel selenoprotein was identified in murine and rat testis RNA using a cDNA amplification with primers derived from the available DNA and protein sequence information (FIG. 3). The sequence of this exon in mouse and rat and the presumable corresponding exon in human and pig is represented in FIG. 3b. It encodes an arginine-rich sequence immediately following the first methionine.

Information as to the function of said sequence was obtained from an experiment wherein two GFP fusion genes were constructed. For the first, the whole alternative exon of the murine sequence, and for the second the sequence subsequent to the second methionine were fused to GFP. After transfection of both constructs into two mammalian cell lines the subcellular localizations of the two fusion proteins were determined by means of GFP fluorescence. Only the largest GFP fusion protein had entered the nucleus indicating that the N-terminal arginine-rich sequence contains a nuclear localization signal (data not shown).

A Northern blot analysis of different tissues of the mouse probed with the Ea sequence of snGPx or with the total sequence of PHGPx (FIG. 4) showed that snGPx is expressed only in the testis. This was confirmed by measuring the distribution of ⁷⁵Se labeled proteins in the murine organism. In this case snGPx was detected only in testis and sperms as has been found earlier for rats (5), wherein this fact indicates a specific function for this selenoenzyme.

Functional Studies of SnGPx

The occurrence of snGPx in late stages of spermatogenesis coincides with the packaging of the DNA with protamines and the stabilization of the resulting highly condensed chromatin by crosslinking of protamine disulfides. (8). This process is induced by reactive oxygen species (ROS) (18) and thus is analogous to glutathione oxidation and peroxide reduction catalyzed by glutathione peroxidases. Together with the finding that the glutathione concentration in spermatids during the late stages of spermatid development markedly decreases (19) this suggests that the selenoenzyme could be capable of using protamine cysteine residues as reducing agents thus acting as a protamine thiol reductase.

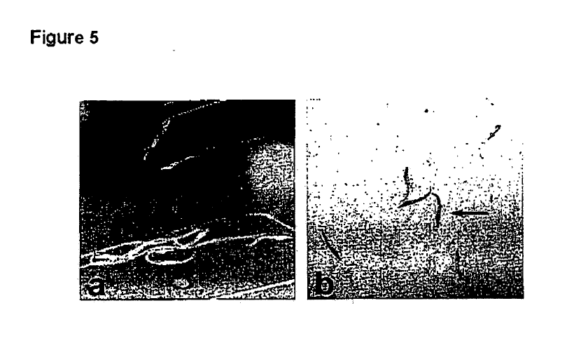

Therefore the sperm nuclear condensation was examined in selenium-deficient rats in which the concentration of selenium and thus of snGPx in CTAB-treated SRS nuclei was decreased to 20 μmol Se/kg of dry weight as compared to a value of 60 μmol Se/kg of dry weight in seleniumadequate animals. The nuclei of both groups were stained with acridine orange making it possible to distinguish between double and single stranded nucleic acids. Since sperm DNA can be denatured by heat only prior to but not after protamine sulfide crosslinking, by means of an acridine orange stain the thiol disuflide state during chromatine condensation of mammalian sperm nuclei can be monitored after the treatment (14). Staining revealed that almost all sperm cells obtained form the vas deferens of the selenium-deficient rats were incompletely condensed (FIG. 5).

In vitro experiments using sperm nuclei of seleniumadequate rats showed that the condensed state was lost during the reduction of protamine disulfides with dithiothreitol and could be reestablished by addition of hydrogen peroxide. The recondensation was blocked by addition of bromosulfophthaleine, an inhibitor of PHGPx (20), or by an excess of another thiol in the form of GSH. It may be concluded from these findings that snGPx is involved in protamine sulfide crosslinking.

The 34 kD selenoprotein found in testis and sperm (5) has now been identified as a specific sperm nuclei glutathione peroxidase having properties similar to those of PHGPx. It is encoded by the PHGPx gene, however, in contrast thereto it is expressed exclusively in testis and starts with an arginine-rich N-terminal sequence encoded by an alternative exon. This sequence contains a nuclear localization signal causing snGPx to be the only selenoprotein able to enter the sperm nucleus.

Another indication as to the significance of this sequence is the fact that is shows more than 50% homology to the sequences of the protamines. Protamines are small basic proteins which are arginine- and cysteine-rich and replacing the histones during sperm maturation. It has been known that they bind to DNA via their polyarginine region and it is very likely that snGPx is associated with DNA in a similar manner.

By our findings that snGPX acts as a protamine thiol peroxidase responsible for the formation of crosslinked protamine disulfides and thus for the stabilization of condensed sperm nuclei, and that a decrease in snGPx levels results in severe defects in chromatin condensation, another important role of selenium has been established. The process of chromatin condensation seems to be important not only for the maturation of the sperm cells but also for the fertility and generation of progeny. In humans, a high correlation between regular sperm condensation and the in vitro fertilization rate has been observed (22). In vitro experiments in mice show that a condensed sperm nucleus having a stable matrix is required to ensure a normal fertilization and embryonic development (23). Thus, the snGPx dependent protamine thiol oxidation appears to play a key role in male fertility and reproduction.

As soon as the sperm reaches the caput epididymis and the condensation process is completed snGPx is partially processed to smaller proteins having the same enzymatic activity but neutral pH values indicating that the basic arginine-rich N-terminal sequence has been lost. Accordingly, snGPx and the processed proteins fulfill two functions: first, the oxidation of protamines by snGPx bound to full length DNA and, second, protection of sperm DNA against oxidative damage by the enzyme and the forms resulting therefrom which can be more efficient in ROS degradation since they are not bound to DNA.

It has been demonstrated in several studies that the quality of human sperms decreases with increasing oxidative damages to the DNA. Since, however, a limited generation of ROS is required for protamine thiol crosslinking DNA damages due to excessive amounts of ROS adversely affect the health of the progeny (24). Consequently, a determination of the snGPx state of the sperm nucleus can be of importance for establishing the sperm quality.

It has been suggested that PHGPx plays a role in chromatin condensation (25) and in the protection of the sperm DNA against oxidative damage (26). However, PHGPx is expressed only in a cytosolic and a mitochondrial form and is mainly membrane-bound in sperms (25). This discrepancy could be solved by the identification of snGPx as the only selenoprotein present in spermatid nuclei and by its characterization as a protamine thiol peroxidase.

It has been known that selenium plays a role in various processes in the male reproductive system. PHGPx acts as a structural component in the mitochondrial capsule (27) and thus is required for flagellum formation whereas in the nuclei snGPx and its processed products are involved in chromatin condensation and in the protection of the germ line against oxidative damage. In addition, selenium has also been reported to be required for testosterone biosynthesis in a manner not identified to date (4). It will be of much interest to find to what level defects in the formation and function of selenoproteins contribute to male fertility disorders.

The following examples merely serve as an illustration and should not be construed as limiting the scope of the present invention.

### EXAMPLES

Animal Experiments

Rats were subjected for several generations either a selenium deficient diet containing 2-5 μg Se/kg or a selenium-adequate diet consisting of the basal diet with 300 μg Se/kg, added in the form of sodium selenite. In the tracer experiments the animals were labeled in vivo by injection of 35 MBq/μm Se) in the form of sodium selenite. The composition of the diet and the treatment of the animals have been described elsewhere (6). In the tracer experiments with mice, adult mice were subjected to the basal diet for two weeks and then labeled by injecting a dose of 3 MBq ⁷⁵Se selenite.

Selenium Determination

The selenium concentrations of tissues and testicular fractions were determined by instrumental neutron activation analysis as described earlier elsewhere (9). In the tracer experiments, the ⁷⁵Se activity in the tissues and tissue fractions was measured by means of a NAI(TI) drill hole detector connected to a four channel analyzer (Canberra, Frankfurt, Germany). The ⁷⁵Se labeled selenoproteins separated by SDS PAGE were examined by means of autoradiography using a light sensitive imaging sheet (Fuji, BAS 1000, Raytest, Straubenhardt, Germany).

Purification of the 34 kD Selenoprotein

Following decapsulation the testes were homogenized in a fourfold volume of a buffer A (20 mM MES, 0.31 M sucrose, 3 mM MgCl₂, 0.1% Triton X-100, 50 mM benzamide HCl, 0.1 mM PMSF, pH 6.0-6.1). After centrifugation at 1000×g the sonication-resistant nuclei (SRS nuclei) of late spermatids were isolated as already described earlier (10) by using a B15 Branson cell disruptor (Heinemann, Schwäbisch-Gmünd, Germany). After incubation with buffers B (1% CTAB, 50 mM Tris, 20 mM DTT, pH 8) and C (1% CHAPS, 50 mM Tris, 20 mM DTT, pH 8), several times washing with buffer D (50 mM Tris, 20 mM DTT, pH 8), extraction with NaCl solution (1 M NaCl in 50 mM Tris, 2% β-mercapto ethanol, pH 8) and centrifugation at 1000×g the supernatant was distilled against aqua dest. and the 34 kD protein was separated from other proteins by means of SDS PAGE.

Protein Identification by MALDI-MS

After SDS PAGE separation the 34 kD band was cut from the gel and a tryptic digestion was carried out as described (11). The digest was extracted with 1% trifluoroacetic acid in acetonitrile. The fragment masses were determined by means of a modified Bruker Reflex III equipped with delayed extraction. Peptide Search (“Protein Identification by Peptide mass Data”, EMBL, Heidelberg) was used for mass identification.

Enzyme Assay

The glutathione peroxidase activity was determined spectrometrically at 340 nm as described (12) by using hydrogen peroxide or t-butylperoxide as substrates. The PHGPx activity was measured by phosphatidylcholine hydroperoxidase which was used as a specific substrate.

Reaction with an Anti-PHGPx Antibody

The 34 kD protein isolated by SDS PAGE was transferred to a PVDF membrane (Biometra, Göttingen, Germany) and probed with a purified anti-rat PHGPx antibody.

N-Terminal Sequencing

The 34 kD protein was isolated by means of SDS PAGE, transferred to a PVDF membrane (Biometra, Göttingen, Germany) and stained with amido black. An N-terminal sequencing was carried out with respect to the bands of interest using an ABI 494 Procise Edman sequencer (PE Biosystems, Foster City, Calif, USA) after the bands had been excised. The amino acids were analyzed in the form of PTH derivatives. The sequencing data obtained in this manner were compared to the sequences in data bases using the “BLAST SEARCH” at NCBI.

cDNA Synthesis by Means of PCR

The 3′ RACE experiments were performed in the GENEAmp system 2400 thermal cycler (Perkin Elmer, Norwalk, Conn, USA). For subsequent sequencing the respective PCR product was cloned into vector pCR2.1-TOPO (Invitrogen, Groningen, Netherlands)

Mouse snGPx: PolyA+RNA was isolated from murine testes using the PolyATRact mRNA isolation system IV (Promega, Madison, Wis, USA). Adapter-bound testis CDNA was synthesized according to the instructions of the manufacturer (Clontech, Palo Alto, Calif.²⁺-, USA). The alternative exon was amplified by means of 3′ RACE using primer PHGPx Ea-fl (GGGACGCTGCAGACAGCGCGGCGGATCC) and Advantage 2 polymerase (Clontech).

Rat snGPx: The data base searches using the sequence of the alternative exon Ea of mouse resulted in an Est clone of rat (Gi: 3399319) which was identical to exon I of PHGPX from rat and to Ea of mouse. The total RNA was isolated from homogenized testes by the acidic guanidinium thiocyanate-phenol-chloroform process (13). An RT-PCR was carried out by means of the Super Script One Step RT-PCR system (Life Technologies, Gibco BRL, Karlsruhe, Germany) using the snGPx specific primer (ATGGGCCGCGCGGCCG) and the primer derived from the rat PHGPX exon 7 (CGGCAGGTCCTTCTCTATCACCTG).

Human snGPx: A presumable Ea for the human 34 kD was found following a comparison of the derived amino acid sequence with the human PHGPX gene (nucleotides 770-964 of gene AF 060973). A 3′ RACE was carried out with adapter-ligated testis cDNA (Clontech) using the snGPx specific primer 1 (ATGGGCCGCGCGGGCGCAGGCTCCC) and primer 2 (CTTGCGACCGGAGATCCACGAATGTCCC) and Advantage 2 polymerase (Clontech). Only the last 14 nucleotides from exon Ea and the transition point to PHGPx were obtained. A search in the Est database resulted in an Est clone (Gi: 2754408) covering the Ea sequence, and in another Est clone (Gi: 6703705) containing the presumable starting methionine.

Pig snGPx: As for the human snGPx a presumable alternative exon was obtained from the pig PHGPx gene (Sus scrofa accession number: X76088) by comparison of the derived amino acids. It encodes an arginine-rich sequence following the presumable translational start. The transition to PHGPX is ambiguous, however, the nucleotide sequence is identical to that of humans.

Northern Blot Analyis

The total RNA was isolated from homogenized tissue using the peqGOLDTriFast (peqLab, Erlangen, Germany). 20 μg of total RNA were applied per lane, blotted onto HybondN⁺(Amersham Pharmacia, Freiburg, Germany) and with the coding region of the alternative exon was probed with PHGPx total cDNA.

Constructs with the Green Fluorescent Protein (GFP)

Two N-terminal GFP fusion proteins were generated by means of overlap PCR. One contained the complete coding region of the alternative exon, the other started at the second presumable translational start. The primer pairs:(GATCTCTAGACCGGCGGGCATGGGCCGCGCG) (GFP-Ea-f1) and (GCTCCTCGCCCTTGCTCACCAAGCCCAGGAACTCGGAGC) (GFP-Ea-r2) as well as (GATCTCAGACTCGCCGGATGGAGCCCATTCC) (GFP-Ea-f2) and GFP-Ea-r2 were used for the amplification of the N-terminal portions for the longer or the shorter version, respectively, by using pMC42 as a template. (GCTCCGAGTTCCTGGGCTTGGTGAGCAAGGGCGAGGAGC) (GFP-F3) and (CCTCTACAAATGTGGTATGG) (GFP-r1) were used to generate the GFP portion using pEGFP-N1 as a template. An overlap PCR was performed by combining the products with the outermost primers. All PCRs were carried out in the Perkin Elmer GENEAmp system 2400 Thermal cycler. The two products were cloned into pCDNA3 via XhiI/XbaI and transiently transfected into NIH3T3 and HeLa cells.

Sperm Chromatin Structural Assay (SCSA)

An abnormal chromatin structure which is evaluated hereby is quantitatively determined by means of flowcytometric measurement of the metachromatic shift of green (dsDNA) to red (denatured, ssDNA) of the acridine orange (A/O) fluorescence. The shift is expressed by the function alpha t (Greek αt) representing the ratio of the red to the total fluorescence intensity (red+green) (Darzynkiewicz et al., 1975) thereby representing the amount of denatured ssDNA with respect to the total cellular DNA. In the SCSA the αt for each spermatozoon in a sample was calculated, and the results were expressed as the mean value (x αt), the standard deviation (SD αt) of the αt distribution and as the percentage of cells having a high αt value referred to as cells outside the main population (COMP) (% COMP αt) representing the cells having an excess of ssDNA. The range of the at values obtained is expressed as a range from 0 to 1024 fluorescence channels.

Sperm Staining with AO

Samples of thawed sperm were diluted with TNE buffer (0.15 M NaCl, 0.01 M Tris-HCl, 1 mM EDTA, pH 7.4) and diluted in polypropylene tubes to a final sperm concentration of approx. 2×10⁶/ml. Aliquots of the diluted sperm (0.2 ml) were deep-frozen immediately in liquid nitrogen steam (LN₂) and then transferred to an ultracold freezer (−80° C.) and stored therein until the FCM examination (SPANO et al., 1999). The samples were codified and a blind FCM analysis was carried out. After thawing on crushed ice the sperm cells were subjected to partial denaturation in situ and stained with AO (MERCK). For this purpose, the sperm samples were mixed with 0.4 ml of detergent solution having a low pH (0.17% Triton X-100, 0.15 M NaCl, and 0.08 N HCl, pH 1.4). After 30 seconds the cells were stained by 1.2 ml of a solution (0.1 M citric acid, 0.2 M Na₂HPO₄, 1 mM EDTA, 0.15 M NaCl, pH 6.0) containing 6 μm/ml of AO. The stained samples were divided in two halves and examined within 3-5 minutes following AO Staining.

Flow-cytometric Analysis

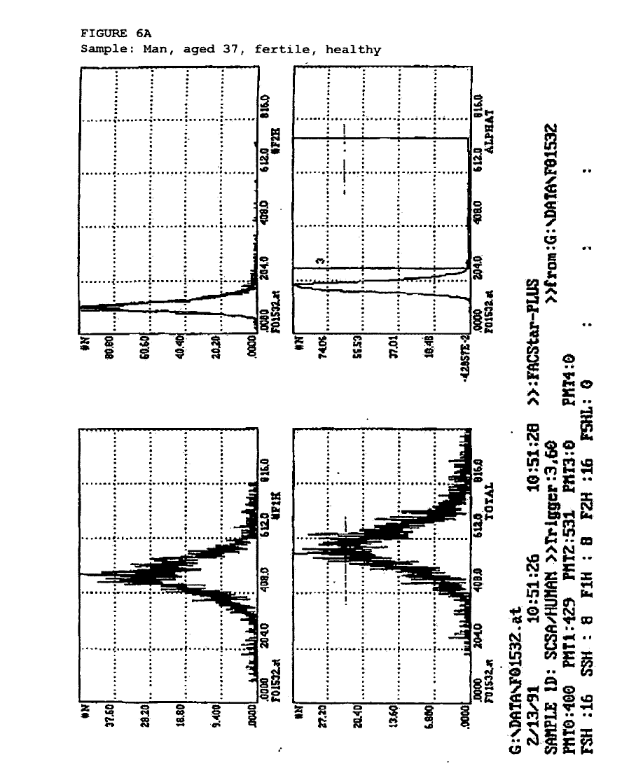

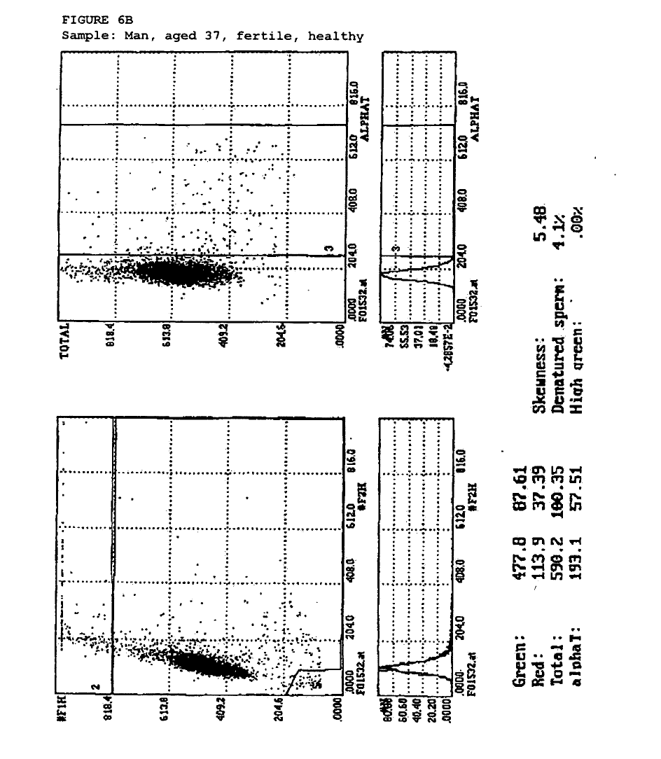

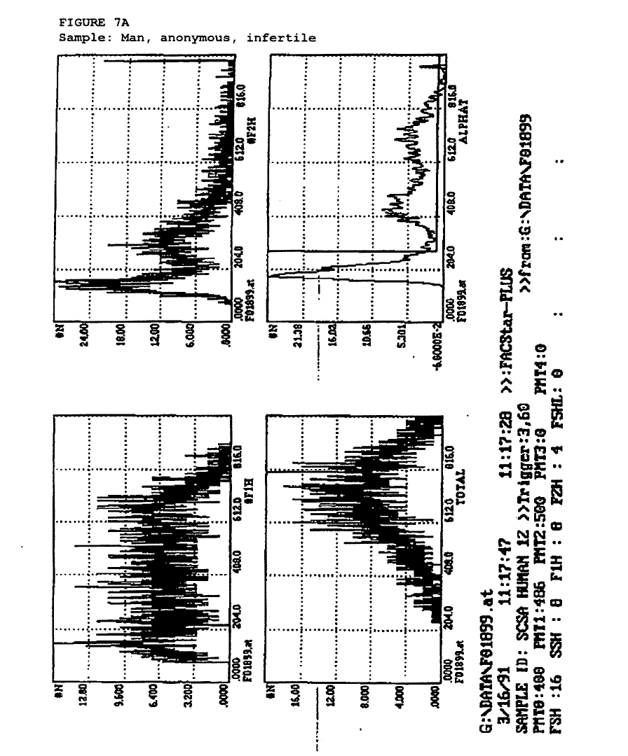

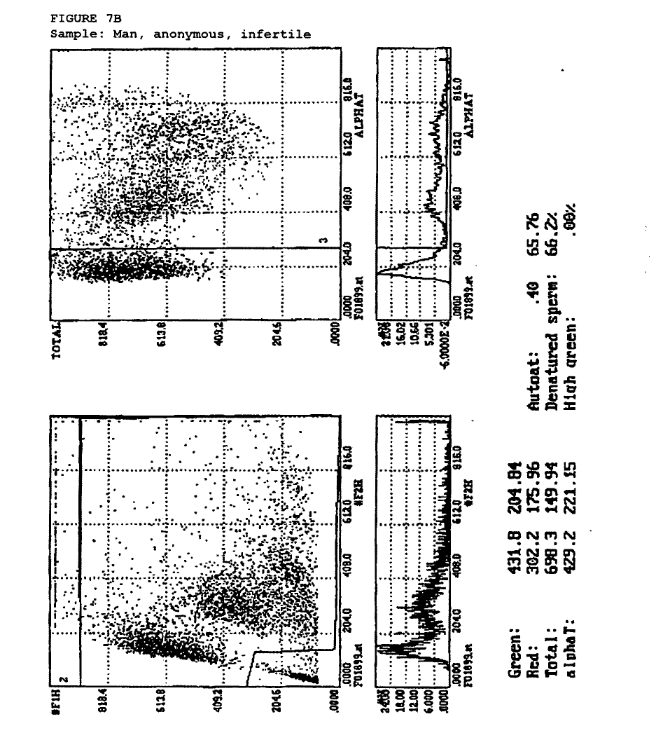

The samples were examined by means of a FACStar Plus flowcytometer (Becton Dickinson Immunochemistry Systems, San José, Calif., USA) equipped with standard optics. After excitation with an Ar ionic laser (Innova 90, Coherent, Santa Clara, Calif., USA) adjusted to 488 nm and 200 mW, A/O intercalated into double stranded DNA shows a green fluorescence (530±30 nm) while AO bound to single stranded DNA fluoresces red (>630 nm). A total of 10,000 results were measured with each sample with a flow rate of about 200 cells/sequences. The fluorescence stability of the flow cytometer was monitored daily using standard beads (Fluoresbrite plain YG 1.0 μm; Polysciences INC., Warrington, Pa, USA). Equivalent instrumental equipment was used for all samples. A distribution diagram analysis of the raw data was performed using Cellquest version 3.1 (Becton Dickinson) wherein each point of the coordinate of the red-green fluorescence intensities evaluates each spermatozoon individually. The results accumulating in the left lower corner correspond to cellular fragments from the sample and were excluded form the examination (see FIGS. 6 and 7 for the evaluation).

### References

1. Behne D., Duk. M., and W. Elger W. (1986) Selenium content and glutathione peroxidase activity in the testis of the maturing rat. J. Nutr. 116,1442-1447.

2. Wu A. S. H., Oldfield J. E., Shull, L. R., and Cheeke, P. R. (1979) Specific effect of selenium deficiency on rat sperm. BioI. Reprod. 20, 793-798.

3. Watanabe T., and Endo, E. (1991) Effects of selenium deficiency on sperm morphology and spermatocyte chromosomes in mice. Mutation Res. 262, 93-99.

4. Behne D., Weiler H., and Kyriakopoulos A. (1996) Effects of selenium deficiency on testicular morphology and function in rats. J. Reprod. Fertil. 106, 291-297.

5. Behne D., Hilmert H., Scheid S., Gessner H., and Elger W. (1988) Evidence for specific selenium target tissues and new biologically important selenoproteins. Biochim. Biophys. Acta 966, 12-21.

6. Behne D., Kyriakopoulos A., Weiss-Nowak C., Kalcklösch M., Westphal C., and Gessner H. (1996) Newly found selenium-containing proteins in the tissues of the rat. Biol. Trace Element Res. 55, 99-110.

7. Behne D., Kyriakopoulos A., Kalckl6sch M., Weiss-Nowak C., Pfeifer H., Gessner H., and Hammel C. ( 1997) Two new selenoproteins found in the prostaric glandular epithelium and in the spermatid nuclei. Biomed. Environ. Sci. 10, 340-345 (1997).

8. Barone L. G., De Lara J., Cummings K. B., and Ward W. S. (1994) DNA organization in human spermatozoa. J. Androl. 15, 139-144.

9. Behne D., Weiss-Nowak C., Kalckl6sch M., Westphal C., Gessner H., and Kyriakopoulos A. (1994) Application of nuclear analytical methods in the investigation and identification of new selenoproteins. Biol. Trace. Elem. Res. 43-45, 287-297.

10. Platz R. O., Meistrich M. L., and Grimes S. R. Jr. (1977) Low-molecular-weight basic proteins in spermatids. Methods Cell. Biol. 16, 297-316.

11. Gevaert K., Verschelde J. L., Puype M., Van Damme J., Goethals M., De Boeck S., and Vandekerckhove J. 1996) Structural analysis and identification of gel-purified proteins, available in the femtomole range, using a novel computer program for peptide sequence assignment, by matrix-assisted laser desorption ionization reflectron time-of-flight-mass spectrometry. Electrophoresis 17, 918-24.

12. Ursini F., Maiorino M., and Gregolin C. (1985) The selenoenzyme phospholipid hydroperoxide glutathione peroxidase. Biochim. Biophys. Acta 839, 62-70.

13. Chirgwin J. M., Przybyla A. F., MacDonald R. J., and Rutter W. J. (1979) Isolation of biologically active ribonucleic acid from sources enriched in ribonuclease. Biochemistry 18, 5294-5299.

14. Kosower N. S., Katayose H., and Yanagimachi R. J. (1992) Thiol-disulfide status and acridine orange fluorescence of mammalian sperm nuclei. J. Androl. 13, 342-348.

15. Rotruck J. T., Pope A. L., Ganther H. E., Swanson A. B., Hafeman D. G., and Hoekstra W. G. (1973) Selenium: biochemical role as a component of glutathione peroxidase. Science 179, 588-590.

16. Takahashi K., Avissar N., Whitin J., and Cohen H. (1987) Purification and characterization of human plasma glutathione peroxidase: a selenoglycoprotein distinct from the known cellular enzyme. Arch. Biochem. Biophys. 256, 677-686.

17. Chu F. F., Doroshow J. H., and Esworthy R. S. (1993) Expression, characterization, and tissue distribution of a new cellular selenium-dependent glutathione peroxidase, GSHPx-GI. J. Biol. Chem. 268, 2571-2576.

18. Holland M. K., Alavarez J. G., and Storey B. T. (1982) Production of superoxide and activity of superoxide dismutase in rabbit epididymal spermatozoa. Biol. Reprod. 27, 1109-1118.

19. Li T. K. (1975) The glutathione and thiol content of mammalian spermatozoa and seminal plasma. Biol. Reprod. 12, 641-646.

20. Tramer F., Rocco F., Micali F., Sandri G., and Panfili E. (1998) Antioxidant systems in rat epididymal spermatozoa. Biol. Reprod. 59, 753-758.

21. Pognay G. C., Corzett M., Weston S., and Balhorn R. (1986) DNA and protein content of mouse sperm. Implications regarding sperm chromatin structure. Exp. Cell Res. 136, 127-136.

22. Claasens O. E., Menkveld R., Franken D. R., Pretorius E., Swart Y., Lombard C. J., and Kruger T. F. (1992) The Acridine Orange test: determining the relationship between sperm morphology and fertilization in vitro. Hum. Reprod. 7, 242-247.

23. Ward W. S., Kimura Y., and Yanagimachi R. (1999) An intact sperm nuclear matrix may be necessary for the mouse paternal genome to participate in embryonic development. Biol. Reprod. 60, 702-706.

24. Aitken R. J. (1999) The Amoroso Lecture. The human spermatozoon -a cell in crisis? J. Reprod. Fertil. 115, 1-7.

25. Godeas C., Tramer F., Micali F., Soranzo M., Sandri G., and Panfili E. (1997) Distribution and possible novel role of phospholipid hydroperoxidase glu tathione peroxidase in rat epididymal spermatozoa. Biol. Reprod. 57, 1502-1508.

26. Hughes C. M., Lewis S. E., McKelvey-Martin V. J., and Thompson W. (1996) A comparison of baseline and induced DNA damage in human spermatozoa from fertile and infertile men, using a modified comet assay. Mol. Hum. Reprod. 2, 613-619.

27. Ursini F., Heim S., Kiess M., Maiorino M., Roveri A., Wissing J., and Flohe L. (1999) Dual function of the selenoprotein PHGPx during sperm maturation. Science 285, 1393-1396.

28. Kelner M. J., and Montoya M. A. (1998) Structural organization of the human selenium-dependent phospholipid hydroperoxidase glutathione peroxidase gene (GPX4): chromosomal localization to 19p13.3. Biochem. Biophys. Res. Commun. 249, 53-55.

29. Ursini, F., Heim, S., Kiess, M., Maiorino, M., Roveri, A., Wissing, J., and Flohe, L. (1999). Dual function of the selenoprotein PHGPX during sperm maturation [see comments], Science 285, 1393- 6.

30. Evenson, D. P., Darzynkiewicz, Z., and Melamed, M. R. (1980). Relation of mammalian sperm chromatin heterogeneity to fertility, Science 210, 1131-3.

31. Tejada, R. I., Mitchell, J. C., Norman, A., Marik, J. J., and Friedman, S. (1984). A test for the practical evaluation of male fertility by acridine orange (AO) fluorescence, Fertil Steril 42, 87-91.

32. Claassens, O. E., Menkveld, R., Franken, D. R., Pretorius, E., Swart, Y., Lombard, C. J., and Kruger, T. F. (1992). The Acridine Orange test: determining the relationship between sperm morphology and fertilization in vitro, Hum Reprod 7, 242-7.

33. Hammadeh, M. E., al-Hasani, S., Stieber, M., Rosenbaum, P., Kupker, D., Diedrich, K., and Schmidt, W. (1996). The effect of chromatin condensation (aniline blue staining) and morphology (strict criteria) of human spermatozoa on fertilization, cleavage and pregnancy rates in an intracytoplasmic sperm injection programme, Hum Reprod 11, 2468-71.

34. Haidl, G., and Schill, W. B. (1994). Assessment of sperm chromatin condensation: an important test for prediction of IVF outcome, Arch Androl 32, 263-6.

35. Hammadeh, M. E., Stieber, M., Haidl, G., and Schmidt, W. (1998). Association between sperm cell chromatin condensation, morphology based on strict criteria, and fertilization, cleavage and pregnancy rates in an IVF program, Andrologia 30, 29-35.

36. Hammadeh, M. E., Askari, A. S., Georg, T., Rosenbaum, P., and Schmidt, W. (1999). Effect of freeze-thawing procedure on chromatin stability, morphological alteration and membrane integrity of human spermatozoa in fertile and subfertile men, Int J Androl 22, 155-62.

37. Hammadeh, M. E., al-Hasani, S., Doerr, S., Stieber, M., Rosenbaum, P., Schmidt, W., and Diedrich, K. 1999). Comparison between chromatin condensation and morphology from testis biopsy extracted and ejaculated spermatozoa and their relationship to ICSI outcome, Hum Reprod 14, 363-7.

38. Evenson D P, Jost L K, Marshall D, Zinaman M J, Clegg E, Purvis K, de Angelis P, Claussen O P. Utility of the sperm chromatin structure assay as a diagnostic and prognostic tool in the human fertility clinic. Hum Reprod. 1999 Apr; 14(4): 1039-49

39. Knopp E A, Arndt T L, Eng K L, Caldwell M, LeBoeuf R C, Deeb S S, O'Brien K D. Murine phospholipid hydroperoxide glutathione peroxidase: cDNA sequence, tissue expression, and mapping. Mamm Genome. 1999 Jun; 10(6):601-5.

40. Brigelius-Flohe R, Aumann K D, Blocker H, Gross G, Kiess M, Kloppel K D, Maiorino M, Roveri A, Schuckelt R, Usani F, et al. Phospholipidhydroperoxide glutathione peroxidase. Genomic DNA, cDNA, and Deduced amino acid sequence. J Biol Chem. 1994 Mar 11;269(10):7342-8.

41. Kelner M J, Montoya M A. Structural organization of the human selenium-dependent phospholipid hydroperoxide glutathione peroxidase gene (GPX4): chromosomal localization to 19p13.3. Biochem Biophys Res Commun. 1998 Aug. 10;249(1):53-5.

What is claimed is:

1. A nucleic acid encoding a phospholipid hydroperoxide glutathione peroxidase (PHGPX) like selenoprotein containing exons 2-7 of the PHGPX gene and one alternative exon within the first intron of the PHGPx gene.

2. The nucleic acid according to claim 1 encoding a mammalian selenoprotein.

3. The nucleic acid according to claim 2 encoding a human selenoprotein wherein the alternative exon comprises the sequence represented in SEQ ID NO: 1 or a portion thereof encoding a biologically active peptide.

4. The nucleic acid according to claim 2 encoding a murine selenoprotein wherein the alternative exon comprises the sequence represented in SEQ ID NO: 2 or a portion thereof encoding a biologically active peptide.

5. The nucleic acid according to claim 2 encoding a rat selenoprotein wherein the alternative exon comprises the sequence represented in SEQ ID NO: 3 or a portion thereof encoding a biologically active peptide.

6. The nucleic acid according to claim 2 encoding a porcine selenoprotein wherein the alternative exon comprises the sequence represented in SEQ ID NO: 4 or a portion thereof encoding a biologically active peptide.

7. An alternative exon within the first intron of the PHGPX gene comprising the sequence represented in SEQ ID NO: 1 or a portion thereof encoding a biologically active peptide.

8. An alternative exon within the first intron of the PHGPX gene comprising the sequence represented in SEQ ID NO: 2 or a portion thereof encoding a biologically active peptide.

9. An alternative exon within the first intron of the PHGPX gene comprising the sequence represented in SEQ ID NO: 3 or a portion thereof encoding a biologically active peptide.

10. An alternative exon within the first intron of the PHGPx gene comprising the sequence represented in SEQ ID NO: 4 or a portion thereof encoding a biologically active peptide.

11. A primer for the amplification of the alternative exon according to claim 1.

12. Primers for the amplification of the alternative exon according to claim 3 which primers have the sequences according to SEQ ID NO: 5 and 6.

13. A mammalian selenoprotein encoded by the nucleic acid according to claim 1 or by an alternative exon of one of SEQ ID NO: 1, 2, 3 or 4 or a portion thereof encoding a biologically active peptide.

14. A human selenoprotein according to claim 13 the N-terminal sequence of which is defined by SEQ ID NO: 7 or by homologs or fragments thereof retaining a biological activity.

15. A murine selenoprotein according to claim 13 the N-terminal sequence of which is defined by SEQ ID NO: 8 or by homologs or fragments thereof retaining a biological activity.

16. A rat selenoprotein according to claim 13 the N-terminal sequence of which is defined by SEQ ID NO: 9 or by homologs or fragments thereof retaining a biological activity.

17. A porcine selenoprotein according to claim 13 the N-terminal sequence of which is defined by SEQ ID NO: 10 or by homologs or fragments thereof retaining a biological activity.

18. A peptide as defined by SEQ ID NO: 7 or by homologs or fragments thereof retaining a biological actiity.

19. A peptide as defined by SEQ ID NO: 8 or by homologs or fragments thereof retaining a biological activity.

20. A peptide as defined by SEQ ID NO: 9 or by homologs or fragments thereof retaining a biological activity.

21. A peptide as defined by SEQ ID NO: 10 or by homologs or fragments thereof retaining a biological activity.

22. An expression vector comprising the nucleic acid according to claim 1 or an alternative exon of one of SEQ ID NO: 1, 2, 3 or 4 or a portion thereof encoding a biologically active peptide.

23. A host cell transformed by a vector according to claim 22.

24. A method of screening for in vitro determination of the fertility of a mammal comprising the following steps:
(a) isolating sperm DNA
(b) amplifying the alternative exon of the PHGPx gene by PCR
(c) sequencing the amplified gene segments
(d) examining the match of the amplified gene segments with the nucleic acid of the alternative exon according to claim 1.

25. The method of screening according to claim 24 wherein the male fertility of humans is determined and the match of the amplified gene segments with the nucleic acid of the alternative exon according to SEQ ID NO: 1 is examined.

26. The method of screening according to claim 24 wherein the male fertility of the mouse is determined and the match of the amplified gene segments with the nucleic acid of the alternative exon according to SEQ ID NO: 2 is examined.

27. The method of screening according to claim 24 wherein the male fertility of the rat is determined and the match of the amplified gene segments with the nucleic acid of the alternative exon according to SEQ ID NO: 3 is examined.

28. The method of screening according to claim 24 wherein the male fertility of the pig is determined and the match of the amplified gene segments with the nucleic acid of the alternative exon according to SEQ ID NO: 4 is examined.

29. The method according to claims 24-28 wherein prior to screening the sperms are tested for nuclear condensation by means of a staining assay.

30. The method according to claim 29 wherein staining is performed by means of acridine orange.

31. An antibody binding specifically to one of the amino acid sequences according to claims 13.

32. The antibody according to claim 31 which is a monoclonal antibody.

33. The antibody according to claims 31 or 32 wherein the antibody is bound to a chemotherapeutical agent or a toxic agent and/or an imaging agent.

34. A hybridoma producing a monoclonal antibody having a binding specificity to any of the amino acids according to claims 13.

35. A recombinant non-human mammal wherein the alternative exon according to claim 1 has been inactivated.

36. A recombinant mouse wherein the nucleic acid according to SEQ ID NO: 2 has been inactivated.

37. A test kit including antibodies according to claims 31-33.

38. A test kit including primers according to claims 11 and 12.

39. A composition comprising an effective amount of a protein/peptide according to any of claims 13 optionally in combination with a pharmaceutically acceptable carrier.

40. A diagnostic method, in which the male infertility is determined by means of a test kit of claim 37, 38 or by means of the composition according to claim 39.

41. An in vivo method for treating a male patient suffering from infertility comprising administering an therapeutically effective amount of the composition of claim 39 to a patient in need of such treatment.

42. The method of claim 41, wherein the administration is performed by direct injection into the testicle.

43. An in vitro method for treating a male patient suffering from infertility comprising treating sperms in vitro with the composition of claim 39.

44. A method for the generation of an essentially pure selenoprotein/peptide comprising transforming a host cell with a vector according to claim 22, culturing the host cell under conditions enabling an expression of the sequence by the host cell, and isolating the peptide from the host cell.
